SEQUENCE LISTING (12 sequences)

SEQ ID NO: 1 - RNA, 19 nt, synthetic construct
  source 1..19: mol_type = other RNA; note = S6 sense strand
cacacacgtggtccgtggt

SEQ ID NO: 2 - RNA, 19 nt, synthetic construct
  source 1..19: mol_type = other RNA; note = S6 antisense strand
accacggaccacgtgtgtg

SEQ ID NO: 3 - RNA, 21 nt, synthetic construct
  source 1..21: mol_type = other RNA; note = S1 sense strand
ctataaaagcggtccccgctt

SEQ ID NO: 4 - RNA, 21 nt, synthetic construct
  source 1..21: mol_type = other RNA; note = S1 antisense strand
gcggggaccgcttttatagtt

SEQ ID NO: 5 - RNA, 21 nt, synthetic construct
  source 1..21: mol_type = other RNA; note = S2 sense strand
cgaatggccaggcccgcgctt

SEQ ID NO: 6 - RNA, 21 nt, synthetic construct
  source 1..21: mol_type = other RNA; note = S2 antisense strand
gcgcgggcctggccattcgtt

SEQ ID NO: 7 - RNA, 21 nt, synthetic construct
  source 1..21: mol_type = other RNA; note = S3 sense strand
attcgcgacccgaagctgctt

SEQ ID NO: 8 - RNA, 21 nt, synthetic construct
  source 1..21: mol_type = other RNA; note = S3 antisense strand
gcagcttcgggtcgcgaattt

SEQ ID NO: 9 - RNA, 21 nt, synthetic construct
  source 1..21: mol_type = other RNA; note = S4 sense strand
ccgagacccgtttggacactt

SEQ ID NO: 10 - RNA, 21 nt, synthetic construct
  source 1..21: mol_type = other RNA; note = S4 antisense strand
gtgtccaaacgggtctcggtt

SEQ ID NO: 11 - RNA, 21 nt, synthetic construct
  source 1..21: mol_type = other RNA; note = S5 sense strand
ccttcgatccgagacccgttt

SEQ ID NO: 12 - RNA, 21 nt, synthetic construct
  source 1..21: mol_type = other RNA; note = S5 antisense strand
acgggtctcggatcgaaggtt

(19) United States
(12) Patent Application Publication
(10) Pub. No.: US 2025/0388898 A1
(43) Pub. Date: Dec. 25, 2025

(54) SMALL ACTIVATING RNA (saRNA) CAPABLE OF ACTIVATING CCAAT ENHANCER BINDING PROTEIN ALPHA (CEBPA) GENE, AND DELIVERY SYSTEM AND USE THEREOF

(71) Applicant: Guangzhou Medical University, Guangzhou (CN)

(72) Inventors: Lingmin ZHANG, Guangzhou (CN); Shunqing TANG, Guangzhou (CN); Yinshan LIN, Guangzhou (CN); Zhouyikang ZHENG, Guangzhou (CN); Jianye ZHANG, Guangzhou (CN); Dazhi ZHOU, Guangzhou (CN)

(21) Appl. No.: 18/886,478

(22) Filed: Sep. 16, 2024

(30) Foreign Application Priority Data
Sep. 18, 2023 (CN) 202311202583.1

(51) Int. Cl. C12N 15/113; A61K 9/51; A61K 38/17; A61P 29/00; B82Y 5/00

(52) U.S. Cl. CPC C12N 15/113; A61K 9/5169; A61K 9/5176; A61K 9/5192; A61K 38/1709; A61P 29/00; B82Y 5/00; C12N 2310/11

(57) ABSTRACT

A small activating RNA (saRNA) capable of activating a CCAAT enhancer binding protein alpha (CEBPA) gene, and a delivery system and use thereof are provided, where sense and antisense strands of the saRNA have nucleotide sequences set forth in SEQ ID NO: 1 to SEQ ID NO: 2, respectively. A biomimetic nano-delivery system for targeted delivery of the saRNA is a biomimetic nanoparticle formed by composite nanoparticle coated by a biomembrane of an inflammatory effector cell, where the composite nanoparticle is formed by histone loading with the saRNA capable of activating the CEBPA gene. The biomimetic nanoparticle can inherit antigens and related membrane functions of the inflammatory effector cell and specifically accumulate at the sites of inflammatory lesions, which enables the biomimetic nanoparticle to not only have a longer circulation time in vivo, but also show inflammatory tropism.

### CROSS REFERENCE TO RELATED APPLICATION

This patent application claims the benefit and priority of Chinese Patent Application No. 202311202583.1 filed with the China National Intellectual Property Administration on Sep. 18, 2023, the disclosure of which is incorporated by reference herein in its entirety as part of the present application.

### REFERENCE TO SEQUENCE LISTING

A computer readable XML file entitled “GWP20240705047”, that was created on Aug. 22, 2024, with a file size of about 13,643 bytes, contains the sequence listing for this application, has been filed with this application, and is hereby incorporated by reference in its entirety.

### TECHNICAL FIELD

The present disclosure belongs to the technical field of biomedicine, and in particular relates to a small activating RNA (saRNA) capable of activating a CCAAT enhancer binding protein alpha (CEBPA) gene, and a delivery system and use thereof.

### BACKGROUND

Acute lung injury (ALI) is a clinical syndrome with an extremely complex etiology, with pathological characteristics being the excessive accumulation and activation of neutrophils in lung tissue. The treatment of ALI mainly includes mechanical respiratory support therapy and glucocorticoid therapy. Although treatment methods are constantly updated and improved, studies have shown that drug treatment is not highly effective in reducing the mortality rate of clinical ALI. Moreover, when ALI develops into the terminal stage, there is a high probability of causing acute respiratory distress syndrome (ARDS), leading to more serious respiratory diseases. Therefore, it is urgent to develop novel drugs or new strategies to deal with the ALI and prevent same from developing into more serious diseases.

Gene therapy has become a new strategy for treating various diseases such as tumors and inflammation. The gene therapy mainly treats a target disease precisely by modifying the genetic genes at a molecular level, achieving the effect of source treatment and efficient treatment. At present, gene therapy has blossomed in the treatment of many diseases, and increasing gene therapy drugs have been approved for clinical use, thereby allowing patients to see a bright prospect. For example, nucleic acid drugs are highly targeted to specific genes, and are more accurate and safer to provide better therapeutic protection for the human body compared to traditional drugs. Gene activation, also known as RNA activation (RNAa), regulates genes through endogenous pathways. Specifically, the RNAa is a phenomenon of using exogenous or endogenous dsRNA to reach the location of a specific target gene through transgenesis, transposon, or viral infection, such that the specific target gene in the cell is activated and expressed. Small activating RNA (saRNA) treatment differs from other RNA-based treatments in that it can upregulate rather than inhibit the expression of therapeutic targets. The saRNA is one of small-molecule double-stranded RNA (dsRNA) with the characteristics of targeting the promoter of a target gene. In addition, the saRNA has a gene activation function and shows the characteristic of upregulating an expression level of the target gene. However, there are currently no reports on saRNA activating specific genes to polarize pro-inflammatory M1 macrophages into anti-inflammatory M2 macrophages, thereby achieving targeted treatment of inflammatory sites.

In addition, a way to avoid an influence of the complex environment in the human body while effectively delivering nucleic acid drugs to the target site remains a technical challenge in gene regulation therapy. Therefore, it is necessary to deliver nucleic acid drugs with efficient carriers to enhance a delivery efficiency and prolong a retention time of the nucleic acid drugs in vivo, thus achieving better therapeutic effects. Histones are endogenous proteins derived from organisms and are alkaline proteins in eukaryotic cell chromatin and prokaryotic cells. The histones as delivery carriers have the advantages of low toxicity and immunogenicity as well as desirable biocompatibility. Accordingly, the histones are ideal delivery carriers of nucleic acid drug that can protect the nucleic acid drugs from the complex environment in human body. However, the complexity of an interaction between nanoparticles in vivo makes it difficult to achieve efficient and precise delivery of the nucleic acid drugs into target sites. Nanoparticles are modified using bionics to adapt to the in vivo environment and can accurately deliver the nucleic acid drugs. By imitating the specificity and selectivity of cells through bionics, the nanoparticles are combined with cell membranes to develop bionic nanotechnology by taking the advantages of both nanoparticles and cell membranes. Biomimetic nanoparticles are coated on their surface with a layer of natural cell membrane, which can directly replicate the specific functions of source cells, thereby effectively possessing the unique characteristics of membrane source cells, such as long circulation in vivo and the function of targeting related diseases. As a result, the biomimetic nanoparticles can make up for the shortcomings of delivery carriers and exhibit great development prospects. It has become a research hotspot to effectively solve the problem of histones in saRNA delivery. In view of this, there is an urgent need to develop a targeted delivery system for saRNA that can provide an effective means for treatment targeting inflammatory sites.

### SUMMARY

Therefore, the objective of the present disclosure is to overcome the defects in the prior art and find an saRNA capable of activating a CEBPA gene. The saRNA can be targeted and delivered to an inflammatory site through a biomimetic nano-delivery system to upregulate an expression level of the CEBPA gene, so as to induce the polarization of M1 macrophages into M2 macrophages, thereby achieving an effect of treating a pulmonary inflammation.

A first aspect of the present disclosure provides an saRNA capable of activating a CEBPA gene, where a sense strand of the saRNA has the nucleotide sequence set forth in SEQ ID NO: 1, and an antisense strand of the saRNA has the nucleotide sequence set forth in SEQ ID NO: 2.

A second aspect of the present disclosure provides a biomimetic nano-delivery system for targeted delivery of an saRNA, where the biomimetic nano-delivery system is a biomimetic nanoparticle of a composite nanoparticle coated by a biomembrane of an inflammatory effector cell, and the composite nanoparticle is prepared from a saRNA loaded histone, where the saRNA is capable of activating a CEBPA gene,
the histone is one or a combination of two or more selected from the group consisting of histone H1, histone H2A, histone H2B, histone H3, and histone H4.

In some embodiments, the biomembrane is one or a combination of two or more selected from the group consisting of a neutrophil membrane, an M1 macrophage membrane, a bone marrow-derived mesenchymal stem cell (BM-MSC) membrane, an umbilical vein-derived mesenchymal stem cell (UV-MSC) membrane, and an erythrocyte membrane.

In some embodiments, the histone is the histone H1, and the biomembrane is the neutrophil membrane.

In some embodiments, the biomembrane and the composite nanoparticle are added at a mass ratio of (1-30):1, preferably (2-10):1, more preferably (3-8):1, more preferably (4-6):1, and furthermore preferably (4.5-5.5):1; and/or,
the histone and the saRNA are added at a mass ratio of (10-50):1, preferably (10-30):1, more preferably (15-25):1, more preferably (17-23):1, and furthermore preferably (19-21):1.

In some embodiments, the composite nanoparticle has a particle size of 100 nm to 300 nm; and/or,
the biomimetic nanoparticle has a particle size of 150 nm to 350 nm, preferably 250 nm to 300 nm; and/or,
the biomimetic nanoparticle has a Zeta potential of −2 mV to −20 mV, preferably −9.8 mV to −11.5 mV; and/or,
the biomimetic nanoparticle has a polydispersity index (PDI) less than or equal to 0.4, preferably less than or equal to 0.2, and more preferably 0.12 to 0.2.

A third aspect of the present disclosure provides a preparation method of the biomimetic nano-delivery system for targeted delivery of an saRNA, including the following steps:
(1) preparing an saRNA solution and a histone solution;
(2) mixing the saRNA solution and the histone solution prepared in step (1) evenly to allow incubation at a room temperature to obtain a composite nanoparticle; and
(3) preparing a biomembrane solution, and adding the composite nanoparticle prepared in step (2) into the biomembrane solution and mixing evenly to allow incubation at a room temperature to obtain the biomimetic nano-delivery system.

In some embodiments, the saRNA solution has a mass concentration of 0.5 μg/μL to 2 μg/μL.

In some embodiments, the histone solution has a mass concentration of 3 μg/μL to 10 μg/μL.

In some embodiments, the biomembrane solution has a mass concentration of 30 μg/μL to 300 μg/μL, and the biomembrane and the saRNA are at a final concentration ratio of (48-52):1.

A fourth aspect of the present disclosure provides use of the biomimetic nano-delivery system for targeted delivery of a saRNA in preparation of a drug for treating a pulmonary inflammation.

In some embodiments, the pulmonary inflammation includes ALI and/or ARDS.

In the present disclosure, the saRNA can activate the upregulation of CEBPA gene expression in inflammatory cells, induce polarization of M1 macrophages into M2 macrophages, and thus play a role in treating inflammation. In the present disclosure, the saRNA has a desirable gene therapy effect on the pulmonary inflammation and is also suitable for the treatment of inflammation related to ALI or ARDS.

In the present disclosure, the biomimetic nano-delivery system for targeted delivery of a saRNA can deliver the saRNA to the inflammatory sites, thereby upregulating the expression level of the CEBPA gene in the cells at the inflammatory sites, thereby achieving an effect of treating inflammation. The histone nanoparticle (composite nanoparticle) in the biomimetic nano-delivery system is positively charged and can effectively load the saRNA. The biomimetic nano-delivery system uses the histone nanoparticle as a delivery carrier of the saRNA, and has long circulation time in vivo and desirable biocompatibility.

Furthermore, the present disclosure selects the histone H1 and the neutrophil membrane in the biomembrane in appropriate amounts to prepare a biomimetic nano-delivery system suitable for delivering the saRNA, such that the biomimetic nanoparticle can not only have a longer circulation time in vivo, but also show inflammatory tropism. Therefore, the system can effectively target the saRNA to the inflammatory sites in an inflammatory environment, improve an efficiency of saRNA delivery into the inflammatory sites, improve a targeting ability of inflammation, and ensure precise delivery of saRNA to the inflammatory sites. Moreover, the system reduces the toxic side effects of saRNA on normal tissues due to weak targeting.

### BRIEF DESCRIPTION OF THE DRAWINGS

FIG. 1 shows screening analysis diagrams of different histones binding to saRNA in Example 1 of the present disclosure; where FIG. 1A is an analysis diagram of the agarose gel loading rate of different histones; FIG. 1B is a line graph of the particle size analysis of different histones binding to saRNA; and FIG. 1C is a line graph of the potential analysis of different histones binding to saRNA;

FIG. 2 shows identification diagrams of the nanoparticle and biomimetic nanoparticle in Example 2 of the present disclosure; where FIG. 2A is a graph showing the particle size and PDI changes of the nanoparticle HR related to the Histone/saRNA ratio; FIG. 2B is a graph showing the particle size and PDI changes affected by the NM/HR ratio; and FIG. 2C is a potential diagram of HR and NHR nanoparticles;

FIG. 3 shows identification and analysis diagrams of the nanoparticle and biomimetic nanoparticle in Example 3 of the present disclosure; where FIG. 3A is a transmission electron microscopy (TEM) diagram of the nanoparticle HR; FIG. 3B is a particle size and PDI analysis diagram of the nanoparticle HR; FIG. 3C is a TEM diagram of the biomimetic nanoparticle NHR; and FIG. 3D is a particle size and PDI analysis diagram of the biomimetic nanoparticle NHR;

FIG. 4 shows SDS-PAGE gel images of the NHR material and neutrophil membrane in Example 3 of the present disclosure;

FIGS. 5A-5D show effects of the biomimetic nanoparticle in Example 4 of the present disclosure on in vitro cell transfection; where FIGS. 5A-5C are Western Blot analysis of the mRNA expression of proteins iNOS, Arg-1, and CEBPA after the biomimetic nanoparticle NHR is transfected into macrophages; FIG. 5D is a RT-qPCR analysis of the changes in proteins iNOS, Arg-1, and CEBPA after the biomimetic nanoparticle NHR is transfected into macrophages, where Tubulin is an internal reference protein;

FIG. 6 shows the effects of different saRNAs on in vitro cell transfection;

FIGS. 7A-7F show the in vivo biodistribution of the nucleic acid drug in Example 5 of the present disclosure; where FIGS. 7A-7B are live imaging diagrams of a small animal; FIG. 7C is a live imaging diagram of the main organs of a small animal; is a fluorescence quantitative analysis diagram of the experimental samples in different organs in FIG. 7B; FIG. 7E is a fluorescence quantitative analysis diagram of the experimental samples of the mouse lung organ at different blood circulation times; and FIG. 7F is a diagram of retention changes in the long-circulation in vivo of different experimental samples; and

FIGS. 8A-8I show characterization diagrams of the therapeutic effect of lung tissue inflammation after treatment in Example 6 of the present disclosure; where FIGS. 8A-8D are diagrams of the mRNA expression of cytokines IL-6, IL-1β, IL-10, and TGF-β in mouse lung tissue after treatment;

FIGS. 8E-8H are diagrams of the content of cytokines IL-6, IL-1β, IL-10, and TGF-β in mouse lung tissue after treatment; and FIG. 8I is an immunofluorescence section of mouse lung tissue.

### DETAILED DESCRIPTION OF THE EMBODIMENTS

The technical solutions of the present disclosure will be further described below through specific examples. Those skilled in the art should understand that these examples only help understand the present disclosure and should not be regarded as specific limitations to the present disclosure.

Unless otherwise defined, all technical and scientific terms used herein have the same meanings as commonly understood by those skilled in the technical field of the present disclosure. The terms used in the description of the disclosure herein is for the purpose of describing particular examples only and is not intended to be limiting of the disclosure.

### Example 1

Screening was conducted on five histones, histone H1, histone H2A, histone H2B, histone H3, and histone H4, to find materials suitable for binding to saRNA. The five histones were prepared and combined with the saRNA, respectively. The positively charged histones and saRNA could self-assemble to form complex nanoparticles due to the positive and negative electrical interactions, and then experiments were conducted on the characterization of the relevant nanoparticles. The characterization experiments included: encapsulation efficiency experiment of agarose gel, particle size, potential, and PDI measurements of HR nano-composites.

The Sequence of the saRNA was:

sense strand:
(SEQ ID NO: 1)
5′-CACACACGUGGUCCGUGGU-3′;
antisense strand:
(SEQ ID NO: 2)
5′-ACCACGGACCACGUGUGUG-3′.

In addition, the 3′-end of each strand was modified with two conventional TT overhang bases.

Experimental method: 5 μg/μL aqueous solutions of five histones were prepared respectively and 1 μg/μL saRNA nuclease-free aqueous solution was prepared. For each histone, the nucleic acid solution was added to the histone solution at mass ratios (w/w) of Histone:saRNA at 1:1, 10:1, and 20:1, pipetted and mixed thoroughly, and immediately vortexed for 30 s to mix thoroughly, such that the nucleic acid and histone were fully mixed, then incubated at room temperature for 10 min to obtain HR nanoparticles.

The HR nanoparticles of the five histones were loaded into agarose gel for encapsulation efficiency experiments to detect the loading rates of saRNA in the five histones. The particle size and PDI of each group of HR nanoparticles of five histones were measured using a Malvern particle size analyzer.

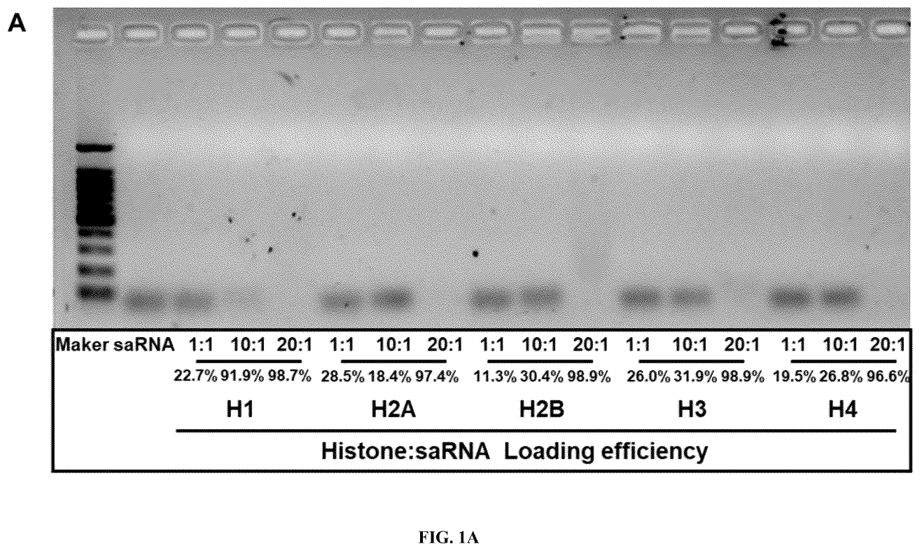

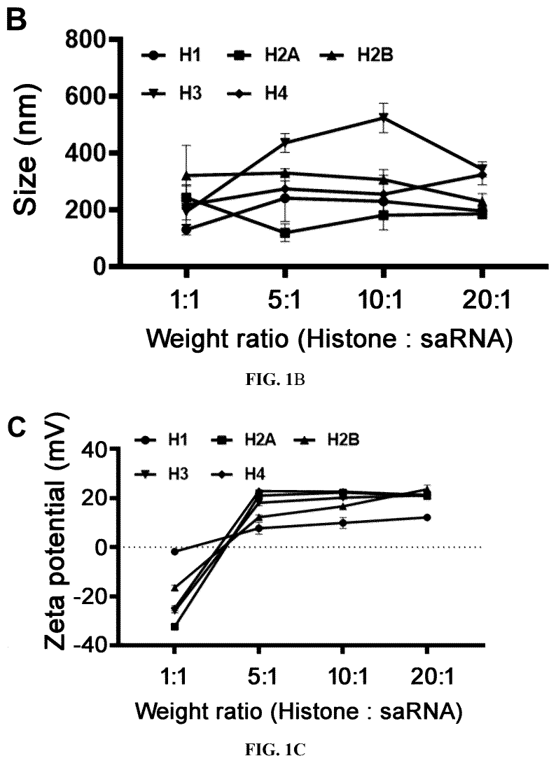

The results were shown in FIG. 1.

As shown in FIG. 1A, after analyzing the loading rates of five histones in agarose gel at different concentrations after binding to saRNA, in the range of Histone:saRNA (1-20):1, the ratio of histones was positively correlated with the loading rate of saRNA. The histone H1 could reach a loading rate of 91.85% at 10:1, and the loading rates of histones H2A, H2B, H3, and H4 at 10:1 were all less than 32%. The selected histone H1 had better binding ability in preparing the saRNA complex nanoparticles than the other four histones.

Referring to FIG. 1B and FIG. 1C, it was seen from the particle size and Zeta potential diagrams of saRNA combined with five histones that the nanoparticles with appropriate proportions of saRNA combined with histone H1 exhibited smaller particle size and the smallest positive Zeta potential than other experimental groups. Moreover, in the subsequent preparation of biomimetic nanoparticle, after comprehensive evaluation of various parameters such as particle size, potential, and PDI, histone H1 combined with neutrophil membrane as selected biomembrane had more advantages than other types of histones. Specific data were omitted.

### Example 2

### Preparation and Identification of Nanoparticle and Biomimetic Nanoparticle

### 1. Particle Size, PDI, and Potential of Nanoparticle (HR)

5 μg/μL Histone (H1 histone) aqueous solution and 1 μg/μL saRNA nuclease-free aqueous solution were prepared, respectively, the nucleic acid solution was added to the histone solution at ratio of Histone:saRNA at 10:1, 20:1, 30:1, 40:1, and 50:1, respectively, pipetted and mixed thoroughly, and immediately vortexed to mix the nucleic acid and histone thoroughly and evenly to obtain the HR nanoparticle. The particle size, PDI, and potential were measured using a Malvern particle size analyzer.

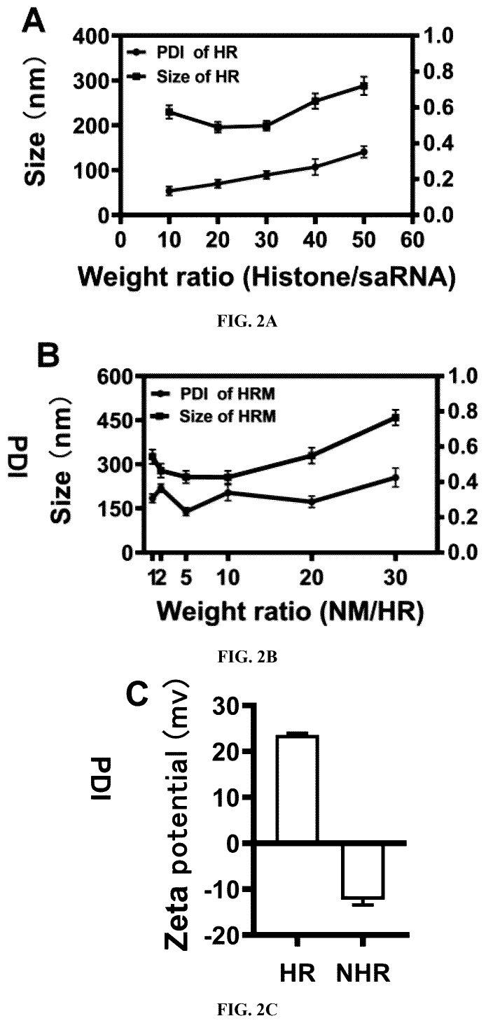

Referring to FIG. 2A and FIG. 2C, when the ratio of H1 histone to saRNA was 20 μg:1 μg, the nanoparticles formed by the interaction of positive and negative charges were relatively uniform, with a nanoparticle size of 201±1.8 nm, a PDI of 0.201, and a Zeta potential of 23.0 mV.

### 2. Particle Size, PDI, and Potential of Biomimetic Nanoparticle (NHR)

A neutrophil membrane was extracted by repeated freezing and thawing, and the neutrophil membrane was treated with a cell disruptor by ultrasound for 1 min, 40 W, on ice for 1 s-on/1 s-off alternately. The neutrophil membrane was added to ddH₂O and mixed to obtain a 50 μg/μL cell membrane solution. The HR nanoparticles prepared with the Histone:saRNA ratio of 20:1 were transferred into the cell membrane solution and prepared with neutrophils. The mass ratios of NM/HR (w/w) were 1, 2, 5, 10, 20, and 30, respectively. A resulting mixture was vortexed for 30 s to mix thoroughly and incubated at room temperature for 10 min to form the NHR nanoparticle by in vitro self-assembly of the neutrophil membrane and the nano-carrier. The particle size, PDI (polydispersity index), and potential were measured using a Malvern particle size analyzer.

Referring to FIG. 2B and FIG. 2C, when the mass ratio of NM/HR in the NHR nanoparticles was 5, the parameters such as particle size, potential, and PDI reached the optimal level, where the particle size was 266±3.3 nm, the PDI was 0.231, and the Zeta potential was −11.2 mV.

### Example 3

This example was used to identify the electron microscopy results of nanoparticles HR and biomimetic nanoparticles NHR and the encapsulation of neutrophil membrane:

### 1. TEM Characterization

10 μL of the HR and NHR nanoparticle samples prepared in Example 2 were added to the electron microscope copper grid and allowed to stand for 10 min. Excess liquid was removed along the edge of the copper grid using absorbent paper, and the copper grid was allowed to drying at room temperature overnight. Electron microscope photographs of each group of samples were taken according to the TEM operating procedures to obtain the morphological characteristics and particle size of the nanoparticles of each sample.

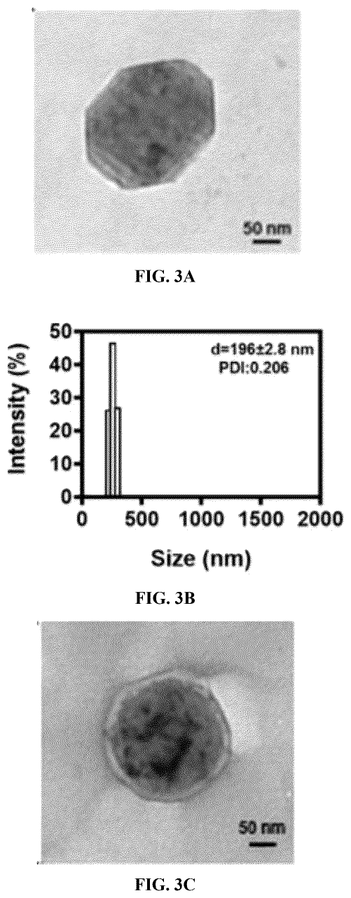

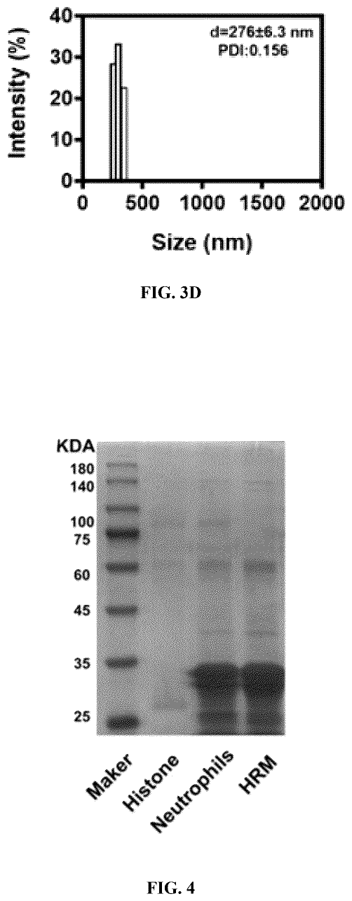

Referring to FIG. 3, it was found that the average particle size of the HR nanoparticles before coating was 196±2.8 nm, the PDI was 0.206, and the Zeta potential was 23.4 mV (FIGS. 3A-3B). The particle size of the NHR nanoparticles coated with cell membrane was 276±6.3 nm, the PDI was 0.156, and the Zeta potential was −9.8 mV (FIGS. 3C-3D). It was seen from the electron microscope image that the neutrophil membrane of this example could evenly coat the periphery of the HR nanoparticles. At the same time, black circles could be clearly seen on the surface of the NHR nanoparticles, which were formed by the neutrophil membrane coating the HR nanoparticles and adhering to the cell membrane. Based on the particle size and potential of the prepared nanomaterials, it was shown that the neutrophil membrane was successfully encapsulated with HR nanoparticles.

### 2. SDS-PAGE Gel Characterization

The obtained NHR nano-composite particles were centrifuged, and the precipitated particles (NHR) were taken out and loaded with the treated neutrophil membrane protein at a mass of 20 μg of membrane protein to allow SDS-PAGE gel electrophoresis. After the electrophoresis, Coomassie Brilliant Blue was conducted for rapid staining. After elution, the specific bands of the sample could be observed.

The experimental results of the gel image in FIG. 4 showed that the bands of the NHR nanoparticles were largely consistent with the bands of the neutrophils, confirming that the NHR nanoparticles retained most of the proteins in the neutrophil membrane.

### Example 4

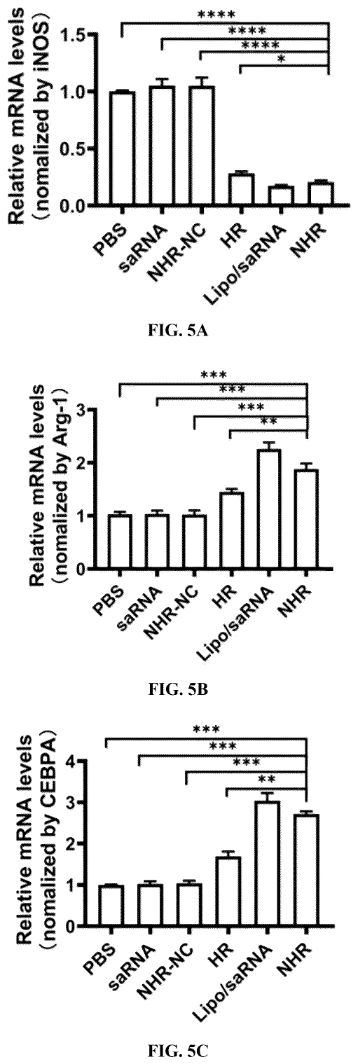

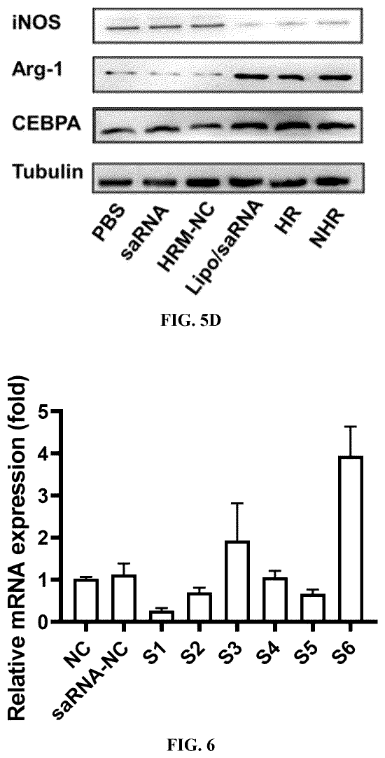

This example was intended to analyze the in vitro cell transfection effect of biomimetic nanoparticles. In this example, a PBS control group, a saRNA group, a NHR-NC group (NHR negative group, composed of Histone, neutrophil membrane, and saRNA-NC), a HR group (histone/saRNA), a Lipo/saRNA group (composed of transfection reagent Lipo3000, saRNA), and an NHR group (saRNA and saRNA-NC in all the above experimental groups were 2 μg) were transfected into M1 macrophages. After 48 h, RT-qPCR and Western Blot were used to analyze the expression levels of cytokines after transfection. The results were shown in FIG. 5. M1 macrophages were treated with different samples: PBS, CEBPA-saRNA, HR, NHR, NHR-NC, and Lipo3000/CEBPA-saRNA transfection for 48 h (CEBPA-saRNA, 2 μg).

Real-time quantitative PCR (RT-PCR) was conducted to evaluate the mRNA levels of inflammatory factors (Arg-1, iNOS, and CEBPA) after NHR treatment. Total RNA was extracted using Trizol reagent, and cDNA was synthesized using SYBR® Green transcriptase kit. Quantitative RT-PCR was conducted using the SYBR GreenER™ qPCR SuperMix Universal Kit. GAPDH was used as a reference gene, and the relative expression of CEBPA gene was quantified by ΔCt method.

Western blot analysis was conducted to analyze the levels of Arg-1, iNOS, and CEBPA after transfection. Proteins were separated on SDS-PAGE and then transferred to PVDF membranes and blocked with 5% skim milk for 1 h at room temperature. The membranes were incubated with primary antibodies (Arg-1, iNOS, and CEBPA) overnight and then goat anti-rabbit secondary antibodies for 1 h. The membrane was exposed by ECL™ Detection Reagent and imaged using the Amersham™ Imager 600 system.

Referring to FIG. 5D, the transfection efficiency of saRNA was analyzed in the Western Blot experiment, and it was found that the samples of the Lipo3000/saRNA, HR, and NHR experimental groups were able to successfully deliver and express saRNA into the cell body, and had a significant transfection effect. Tubulin was used as an internal reference protein, the marker protein iNOS of M1 macrophages was reduced, while the marker protein Arg-1 of M2 macrophages and the target protein CEBPA were increased.

As shown in FIGS. 5A-5C, in the real-time fluorescence PCR experiment, the mRNA of iNOS, a marker protein of M1 macrophages, was significantly reduced after transfection with NHR biomimetic nanoparticles, and the mRNA of marker protein Arg-1 of M2 macrophages and the mRNA of protein CEBPA was significantly upregulated. The above Western Blot analysis results were consistent with the expression of real-time fluorescence PCR results, indicating that the saRNA drug had played a role in activating and upregulating the CEBPA gene, thereby further polarizing M1 macrophages into M2 macrophages.

Multiple saRNA sequences were designed, and after initial screening, 6 saRNA sequences (S1 to S6) were selected as follows:

S1 sense strand:
(SEQ ID NO: 3)
CUAUAAAAGCGGUCCCCGC TT
S1 antisense strand:
(SEQ ID NO: 4)
GCGGGGACCGCUUUUAUAG TT
S2 sense strand:
(SEQ ID NO: 5)
CGAAUGGCCAGGCCCGCGC TT
S2 antisense strand:
(SEQ ID NO: 6)
GCGCGGGCCUGGCCAUUCG TT
S3 sense strand:
(SEQ ID NO: 7)
AUUCGCGACCCGAAGCUGC TT
S3 antisense strand:
(SEQ ID NO: 8)
GCAGCUUCGGGUCGCGAAU TT
S4 sense strand:
(SEQ ID NO: 9)
CCGAGACCCGUUUGGACAC TT
S4 antisense strand:
(SEQ ID NO: 10)
GUGUCCAAACGGGUCUCGG TT
S5 sense strand:
(SEQ ID NO: 11)
CCUUCGAUCCGAGACCCGU TT
S5 antisense strand:
(SEQ ID NO: 12)
ACGGGUCUCGGAUCGAAGG TT
S6 sense strand:
(SEQ ID NO: 1)
CACACACGUGGUCCGUGGU TT
S6 antisense strand:
(SEQ ID NO: 1)
ACCACGGACCACGUGUGUG TT

According to the preparation method of biomimetic nanoparticle (NHR) in Example 2, the corresponding NHR particles were prepared using the PBS control group, saRNA-NC group, S1 to S6 (the saRNA in all the above experimental groups was 2 μg), and then transfected into M1 macrophages. After 48 h, RT-qPCR was conducted to analyze the expression level of CEBPA after transfection. The results were shown in FIG. 6, among which the saRNA-6 sequence (i.e., the saRNA in the present disclosure) had the best effect.

### Example 5

This example aimed to analyze a targeting effect of the biomimetic nanoparticle in animals in vivo.

The experimental samples were divided into saline group, saRNA group, HR group, and NHR group. The samples used Cy5 fluorescently labeled saRNA, and a small animal in vivo imaging device was used to detect the specific distribution of the samples in mice.

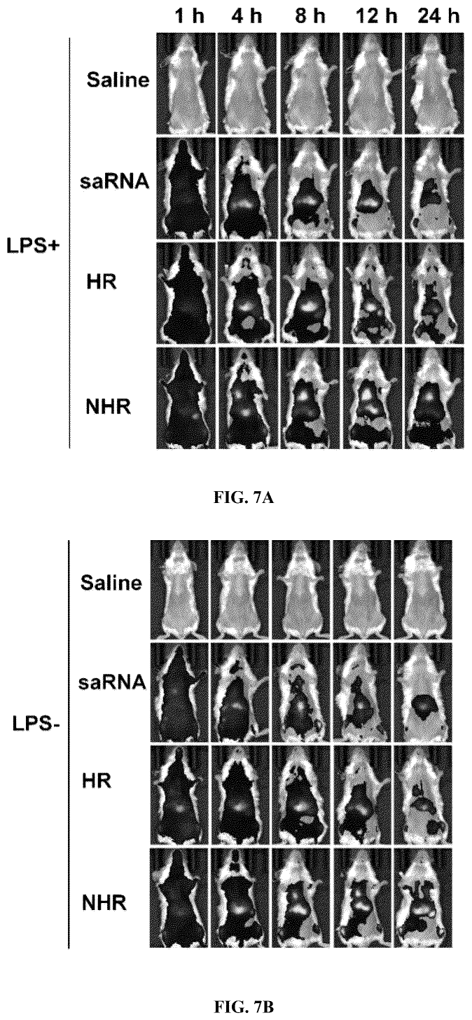

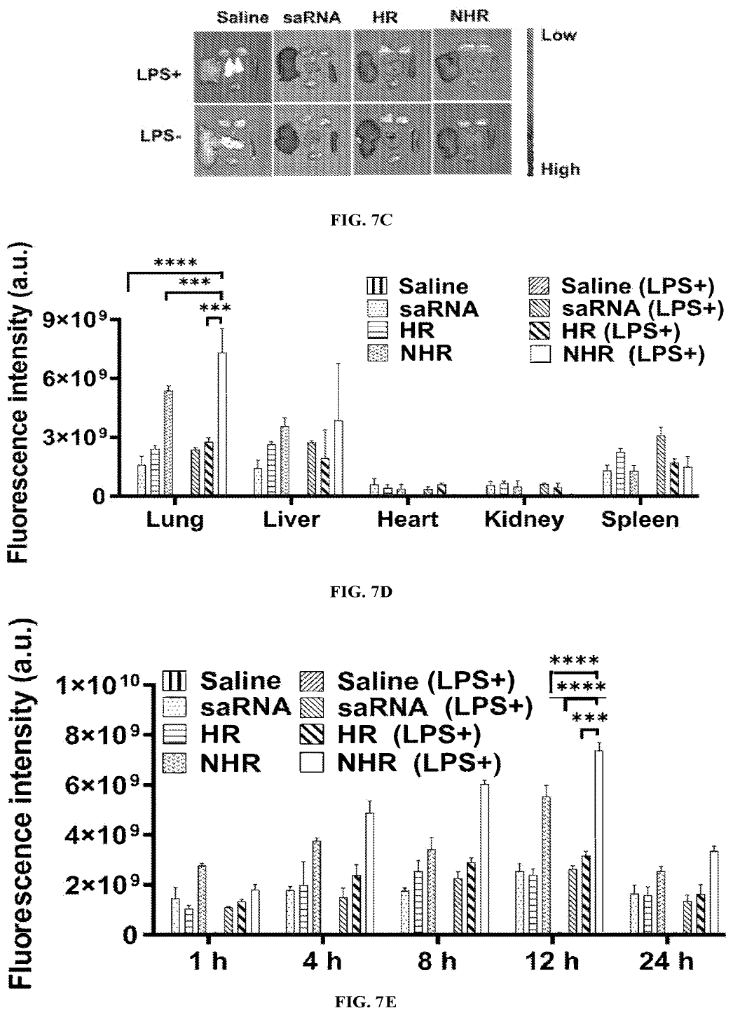

Referring to FIGS. 7A-7B, the results of fluorescence image analysis showed that NHR nanoparticles aggregated in the lungs of inflamed mice, and the fluorescence of the fluorescent dye Cy5-saRNA remained mostly in the lungs during the observation period, demonstrating the specific delivery of saRNA drugs to the inflammatory site.

Referring to FIG. 7C, the fluorescence image showed that the nanoparticles without coating neutrophil membrane mainly accumulate in the liver. After the mice were sacrificed, the main organs were extracted for imaging, including: lungs, liver, heart, kidneys, and spleen. Fluorescence images confirmed that the biomimetic nanoparticles mainly accumulated in the lungs, and the nanoparticles without coating neutrophil membrane accumulated in the liver and spleen.

Meanwhile, referring to FIGS. 7A-7F, the fluorescence intensity of each organ in FIG. 7C after 24 h was processed using Living Image software to obtain quantitative fluorescence data, indicating that the fluorescence value of the NHR nanoparticles coated with the neutrophil membrane reached maximum in the lungs. Referring to FIGS. 7A-7F, through the quantitative fluorescence data of the lung organs in mice, it was found that the NHR nanoparticles coated with neutrophil membrane were more than twice as many as the nanoparticles without coating cell membrane, and the fluorescence signal began to decrease after 12 h. This also demonstrated that nanoparticles camouflaged and coated with neutrophil membrane showed inflammatory tropism in vivo.

Referring to FIG. 7F, the results of the in vivo long circulation experiment showed that the pure Cy5-saRNA in the blood was less than 10% after 24 h of circulation, and was almost completely metabolized. In contrast, the circulation of NHR nanoparticles coated with neutrophil membrane in the blood still had more than 35% retention at 48 h. Meanwhile, the NHR nanoparticles coated with neutrophil membrane were also significantly improved compared with HR nanoparticles without neutrophil membrane, allowing the nanoparticles to circulate in the blood for a longer time.

### Example 6

This example aimed to determine a therapeutic effect of the biomimetic nanoparticle on animal inflammation in vivo.

Gene expression and cytokine expression levels in lung tissue were evaluated after nanoparticle treatment in mice.

### 1. QPCR and ELISA Detection

The blank control group (LPS), saRNA group, NHR-NC group, HR group, and NHR group of experimental samples were set up to treat mice. The dosage of each experimental group was 20 μg/mouse. QPCR and ELISA were conducted to detect and analyze the expression levels of inflammatory factors IL-1β and IL-6 as well as anti-inflammatory factors IL-10 and TGF-β in lung tissues.

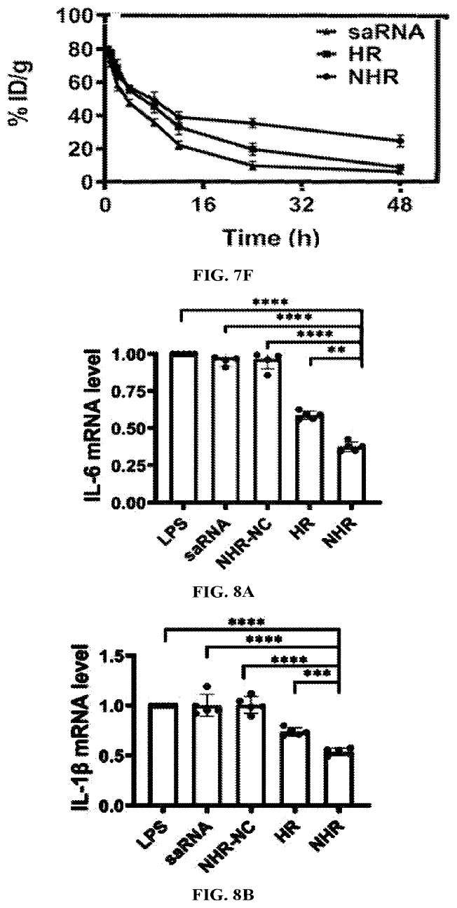

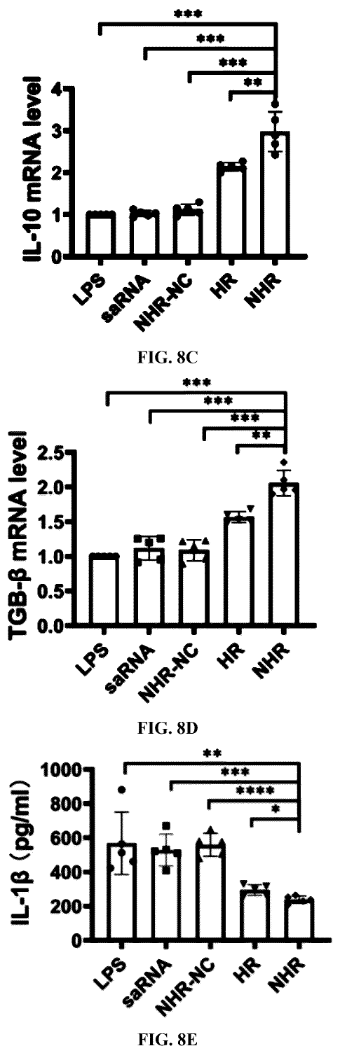

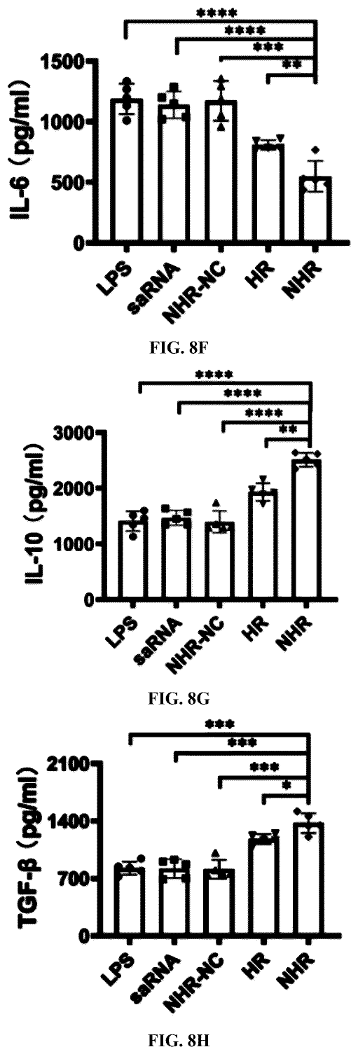

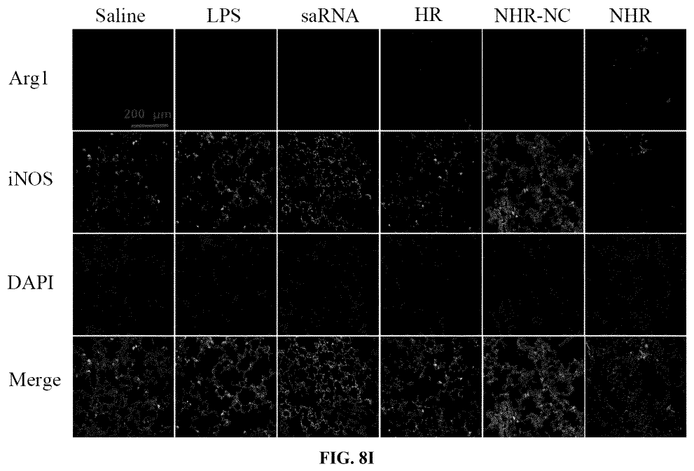

Referring to FIGS. 8A-8D, the results of QPCR analysis showed that the mRNA expression of inflammatory factors such as IL-1β and IL-6 in the lung tissue of mice was significantly downregulated after NHR treatment, and the mRNA expression of anti-inflammatory factors IL-10 and TGF-β was significantly upregulated.

Referring to FIGS. 8E-8H, the results of ELISA test showed that the levels of IL-1β and IL-6 decreased, while the levels of anti-inflammatory factors IL-10 and TGF-β increased after NHR treatment. This confirmed that NHR could effectively inhibit inflammatory factors and promoted anti-inflammatory factors.

The above results showed that the NHR nanoparticles encapsulating the saRNA had a higher inflammatory treatment effect than other treatment groups and the control group. Further, the expression level of inflammatory factors was lower than that of other treatment groups, which more effectively inhibited the occurrence of ALI inflammation.

### 2. Immunofluorescence Detection

The experimental samples of saline group, LPS group, saRNA group, NHR-NC group, HR group, and NHR group were set up to treat mice, and the dosage of each experimental group was 20 μg/mouse. The lung tissue of mice was taken to make immunofluorescence sections, and DAPI was used as a fluorescent dye for cell staining. The infiltration of leukocytes (such as neutrophils and macrophages) in the lungs was observed by confocal fluorescence, while the distribution of iNOS protein (biomarker of M1 macrophages) and Arg-1 protein (biomarker of M2 macrophages) in leukocytes was analyzed.

Referring to the confocal fluorescence analysis of FIG. 8I, the iNOS-positive cells were significantly reduced while the Arg-1-positive cells were significantly increased in the NHR treatment group compared with those in the HR group. These indicated that after NHR nanoparticle treatment, M1 macrophages were polarized into M2 macrophages.

In summary, immunofluorescence and section staining demonstrated that the biomimetic nanoparticle NHR played an important role in the treatment of pneumonia-related diseases.

The above described are merely several embodiments of the present invention. Although these embodiments are described specifically and in detail, they should not be construed as a limitation to the patent scope of the present disclosure. It should be noted that those of ordinary skill in the art can further make several variations and improvements without departing from the concept of the present disclosure, and all of these fall within the protection scope of the present disclosure. Therefore, the protection scope of the present disclosure shall be subject to the protection scope defined by the claims.

What is claimed is:

1. A small activating RNA (saRNA) capable of activating a CCAAT enhancer binding protein alpha (CEBPA) gene, wherein a sense strand of the saRNA has the nucleotide sequence set forth in SEQ ID NO: 1, and an antisense strand of the saRNA has the nucleotide sequence set forth in SEQ ID NO: 2.

2. A biomimetic nano-delivery system for targeted delivery of a saRNA, wherein the biomimetic nano-delivery system is a biomimetic nanoparticle formed by a composite nanoparticle coated with a biomembrane of an inflammatory effector cell, and the composite nanoparticle is prepared from histone and a saRNA capable of activating a CEBPA gene,
the histone is one or a combination of two or more selected from the group consisting of histone H1, histone H2A, histone H2B, histone H3, and histone H4; preferably, the histone is the histone H1, and the histone and the saRNA are added at a mass ratio of (10-50):1, preferably (10-30):1.

3. The biomimetic nano-delivery system for targeted delivery of a saRNA according to claim 2, wherein the saRNA capable of activating the CEBPA gene is the saRNA capable of activating the CEBPA gene, wherein a sense strand of the saRNA has the nucleotide sequence set forth in SEQ ID NO: 1, and an antisense strand of the saRNA has the nucleotide sequence set forth in SEQ ID NO: 2.

4. The biomimetic nano-delivery system for targeted delivery of a saRNA according to claim 2, wherein the biomembrane is one or a combination of two or more selected from the group consisting of a neutrophil membrane, an M1 macrophage membrane, a bone marrow-derived mesenchymal stem cell (BM-MSC) membrane, an umbilical vein-derived mesenchymal stem cell (UV-MSC) membrane, and an erythrocyte membrane; and/or, the histone is the histone H1.

5. The biomimetic nano-delivery system for targeted delivery of a saRNA according to claim 2, wherein the biomembrane and the composite nanoparticle are added at a mass ratio of (1-30):1, preferably (2-10):1, more preferably (3-8):1, and furthermore preferably (4.5-5.5):1; and/or,
the histone and the saRNA are at a dosage ratio of (15-25):1, preferably (19-21):1.

6. The biomimetic nano-delivery system for targeted delivery of a saRNA according to claim 2, wherein the composite nanoparticle has a particle size of 100 nm to 300 nm; and/or,
the biomimetic nanoparticle has a particle size of 150 nm to 350 nm; and/or,
the biomimetic nanoparticle has a Zeta potential of −2 mV to −20 mV, preferably −9.8 mV to −11.2 mV; and/or,
the biomimetic nanoparticle has a polydispersity index (PDI) no more than 0.4, preferably no more than 0.2.

7. A method for preparing the biomimetic nano-delivery system for targeted delivery of a saRNA according to claim 2, comprising the following steps:
(1) preparing a saRNA solution and a histone solution;
(2) mixing the saRNA solution and the histone solution prepared in step (1) evenly to allow incubation at a room temperature to obtain a composite nanoparticle; and
(3) preparing a biomembrane solution, and adding the composite nanoparticle prepared in step (2) into the biomembrane solution and mixing evenly to allow incubation at a room temperature to obtain the biomimetic nano-delivery system.

8. The method for preparing the biomimetic nano-delivery system for targeted delivery of a saRNA according to claim 7, wherein
the biomembrane and the composite nanoparticle are added at a mass ratio of (1-30):1, preferably (2-10):1, more preferably (3-8):1, and furthermore preferably (4.5-5.5):1; and/or,
the histone and the saRNA are added at a mass ratio of (10-50):1, preferably (10-30):1, more preferably (15-25):1, and furthermore preferably (19-21):1.

9. A method for preparing a drug for treating a pulmonary inflammation, comprising conducting the biomimetic nano-delivery system for targeted delivery of a saRNA according to any one of claim 2.

10. The method according to claim 9, wherein the pulmonary inflammation comprises acute lung injury (ALI) and/or acute respiratory distress syndrome (ARDS).

11. The biomimetic nano-delivery system for targeted delivery of a saRNA according to any one of claim 6, wherein the saRNA capable of activating the CEBPA gene is the saRNA capable of activating the CEBPA gene, wherein a sense strand of the saRNA has the nucleotide sequence set forth in SEQ ID NO: 1, and an antisense strand of the saRNA has the nucleotide sequence set forth in SEQ ID NO: 2.

12. The biomimetic nano-delivery system for targeted delivery of a saRNA according to any one of claim 6, wherein the biomembrane is one or a combination of two or more selected from the group consisting of a neutrophil membrane, an M1 macrophage membrane, a bone marrow-derived mesenchymal stem cell (BM-MSC) membrane, an umbilical vein-derived mesenchymal stem cell (UV-MSC) membrane, and an erythrocyte membrane; and/or, the histone is the histone H1.

13. The biomimetic nano-delivery system for targeted delivery of a saRNA according to any one of claim 6, wherein the biomembrane and the composite nanoparticle are added at a mass ratio of (1-30):1, preferably (2-10):1, more preferably (3-8):1, and furthermore preferably (4.5-5.5):1; and/or,
the histone and the saRNA are at a dosage ratio of (15-25):1, preferably (19-21):1.
